(19) United States
(12) Patent Application Publication
(10) Pub. No.: US 2021/0289902 A1
(43) Pub. Date: Sep. 23, 2021

(54) DEVICE AND SYSTEM FOR MEDICINE STORAGE

(71) Applicant: Lifecase, LLC, Florissant, MO (US)

(72) Inventor: Kelly Alexis Baird, Florissant, MO (US)

(73) Assignee: Lifecase, LLC, Florissant, MO (US)

(21) Appl. No.: 17/207,668

(22) Filed: Mar. 20, 2021

Related U.S. Application Data
(60) Provisional application No. 62/992,844, filed on Mar. 20, 2020.

(51) Int. Cl. A45C 11/00; A61J 1/05; A61M 5/00; A61M 5/20; A45C 15/00; G08B 7/06; G08B 21/12

(52) U.S. Cl. CPC A45C 11/00; A61J 1/05; A61M 5/002; A61M 5/20; A45C 15/00; H04W 76/10; G08B 21/12; A45C 2011/002; A45C 2011/007; A45C 2200/05; G08B 7/06

(57) ABSTRACT

The present disclosure relates to a safety device, system, and method for storing medicines by a patient or caretaker without the harm from environmental elements. Medicines that require patients or caretakers to store at non-ambient conditions, such as non-room temperatures, specific humidity, no natural or artificial light, etc., are inconvenient and stressful to carry around during daily activities. In the absence of maintaining the required environmental conditions such as temperature, humidity, light, etc., the medicine(s) become less potent and efficient. Therefore, the invention herein relates to a medicine storage systems and device so that the medicines can be carried during day-to-day activities under required conditions.

### CROSS-REFERENCE TO RELATED APPLICATIONS

This application is a Non-Provisional Application claiming priority of the Provisional U.S. Application No. 62/992,844 filed on Mar. 20, 2020, and is incorporated in entirety herein by references.

### STATEMENT REGARDING FEDERALLY SPONSORED RESEARCH OR DEVELOPMENT

Not applicable.

The present disclosure relates to a safety device, system, and method for storing medicines by a patient or care-taker without the harm from environmental elements.

### BACKGROUND

Medications, including life-saving medicines, require that the patient stores them at specific conditions; without storing under the specific storage conditions and requirements, the medication may become inactivate. Therefore, the medicine, when stored inappropriately, may be ineffective, leading to severe and harmful, in some situations life-threatening, impact. Hence, there is a need for safety devices that stores the medicine under specific required environmental conditions, and such a device is lacking in the market place. This invention provides a safety device, system, and methods for medicine storage by a patient during daily living activities.

### BRIEF DESCRIPTION OF THE INVENTION

Medicines that require patients or care-takers to store at non-ambient conditions, such as non-room temperatures, specific humidity, no natural or artificial light, are inconvenient and stressful to carry around during daily activities. The invention herein relates to a medicine storage systems and device so that the medicines may be carried during day-to-day activities under required conditions.

A system and device herein configured to safely store one or more medicines comprising a hollow jacket with inner compartments, and the compartments are further configured to comprise one or more medicine storage compartments, one or more vacuum, insulating material, heated material, cooled material, light-reflective material, humidifying material, dehumidifying material, or any combination thereof.

### BRIEF DESCRIPTION OF THE INVENTION

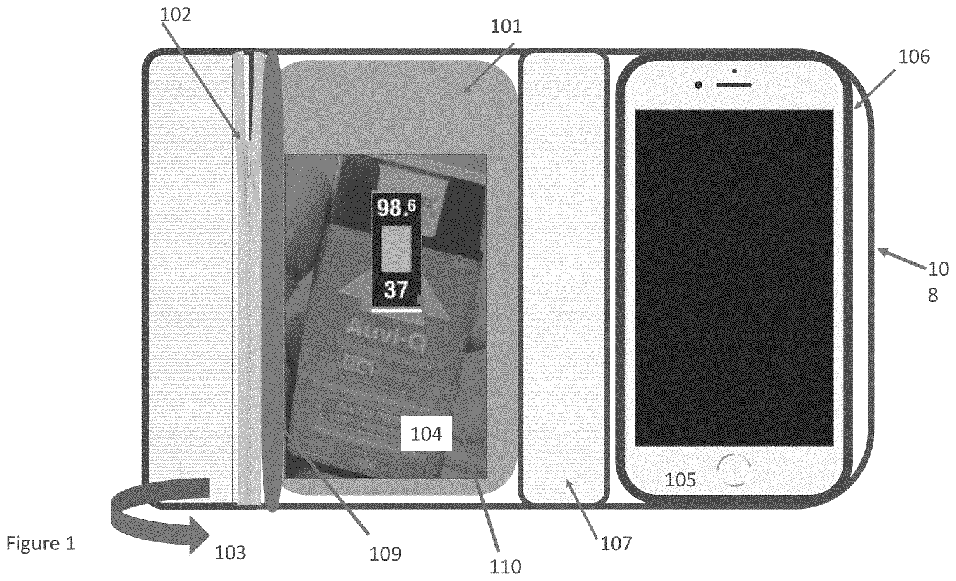

FIG. 1: One View of the Storage Device.

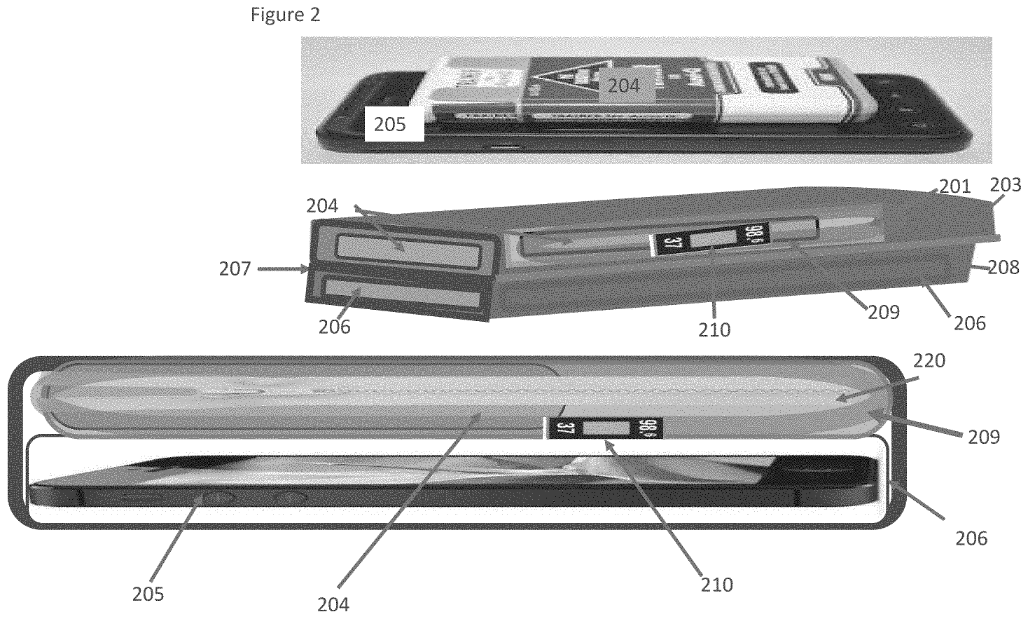

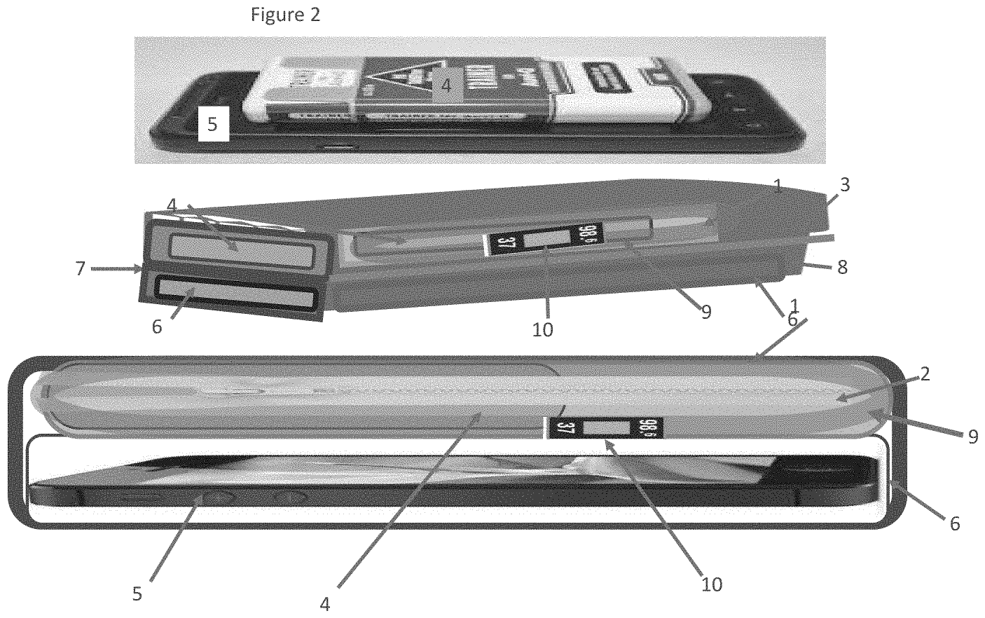

FIG. 2: Side View of Stage Device with Foldable Style.

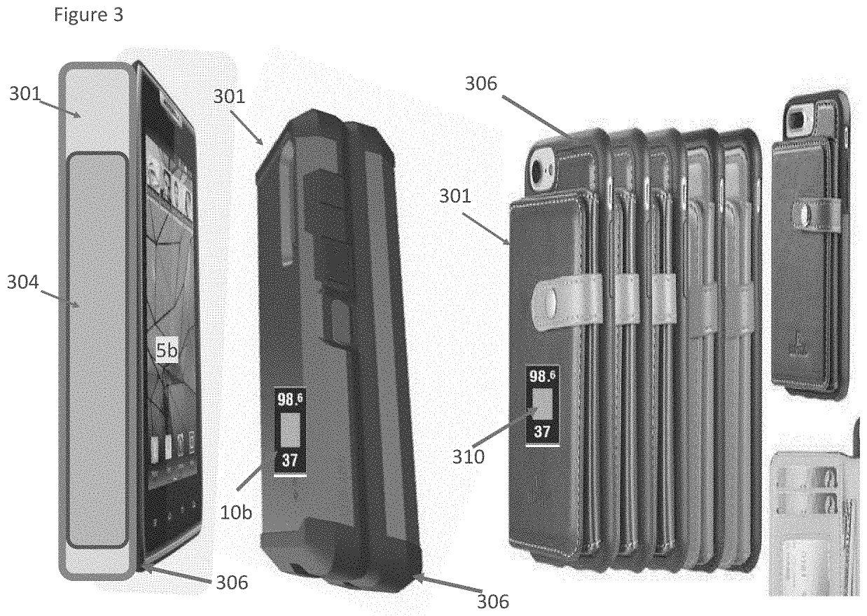

FIG. 3: Storage Device Attachment Option with Side View.

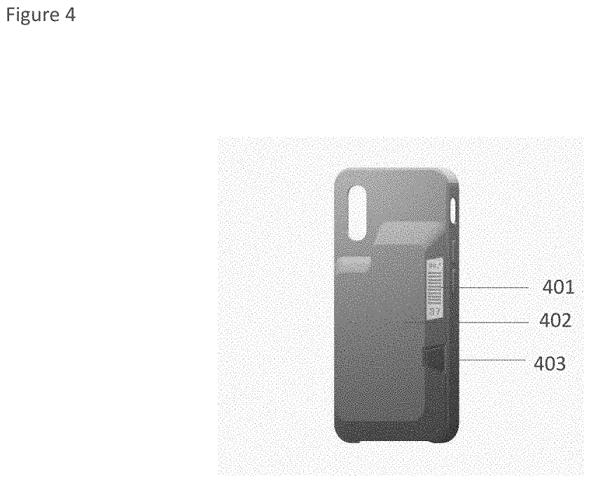

FIG. 4: A prototype of the device and system.

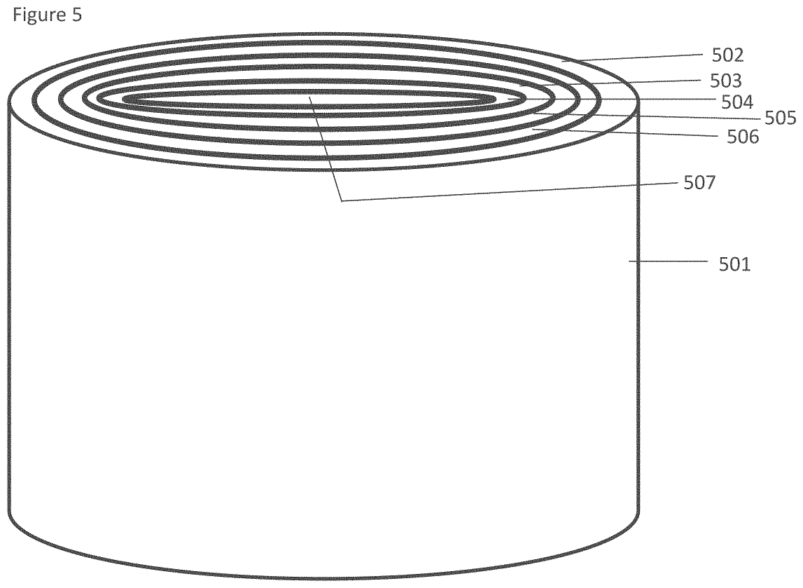

FIG. 5 An assembly of compartment and sub-compartments.

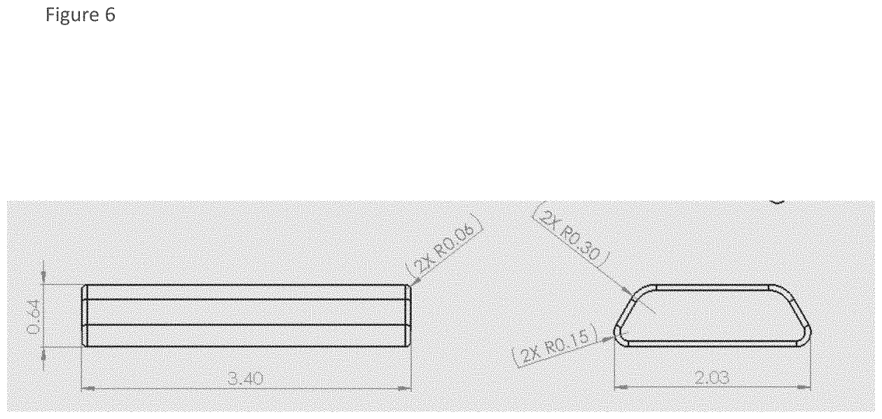

FIGS. 6-9: Embodiments of the prototype.

### DETAILED DESCRIPTION OF THE INVENTION

Examples embodiments are provided so that this disclosure will be thorough and will fully convey the scope to those who are skilled in the art. Numerous specific details are set forth, such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components but do not preclude the presence or addition of one or more other features, integers, steps, operations, elements, components, and/or groups thereof. The method steps, processes, and operations described herein are not to be construed as necessarily requiring their performance in the particular order discussed or illustrated unless specifically identified as an order of performance. It is also to be understood that additional or alternative steps may be employed.

Although the terms first, second, third, etc., may be used herein to describe various elements, components, regions, layers and/or sections, these elements, components, regions, layers and/or sections should not be limited by these terms. These terms may be only used to distinguish one element, component, region, layer, or section from another region, layer, or section. Terms such as “first,” “second,” and other numerical terms when used herein do not imply a sequence or order unless clearly indicated by the context. Thus, a first element, component, region, layer, or section discussed below could be termed a second element, component, region, layer, or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” may encompass both an orientation of above and below. The device may be otherwise oriented (rotated 90 degrees or at other orientations) and the spatially relative descriptors used herein interpreted accordingly.

The preceding summary, as well as the following detailed description of certain embodiments, will be better understood when read in conjunction with the appended figure of experimental data and results. As used herein, an element or step recited in the singular and proceeded with the word “a” or “an” should be understood as not excluding the plural of said elements or steps unless such exclusion is explicitly stated. Furthermore, references to “one embodiment” or “an embodiment” are not intended to be interpreted as excluding the existence of additional embodiments that also incorporate the recited features. Moreover, unless explicitly stated to the contrary, embodiments “comprising” or “having” an element or a plurality of elements having a particular property may include additional such elements not having that property. When a definition is provided herein, it supersedes any other meaning or definition.

As used herein, the term “outer jacket” means the outermost part or the cover of the device that has one or more compartments.

As used herein, the term “compartment” means a compartment, channels, or section of for housing the medicine or other components details herein.

As used herein, the term “device” means an apparatus.

As used herein, the term “conditions/environment-sensitive” means specific environmental conditions such as particular temperature, humidity, light exposure, etc.

As used herein, the term “safely stored” means stored under specific conditions of temperature, humidity, light exposure, etc.

As used herein, the term “wearable” means that which may be attached to or worn by a human.

As used herein, the term “replaceable inserts” means replaced by the same or another with better functional insert.

Several environment-sensitive conditions such as temperatures-, humidity-, pressure-, light-sensitive (natural or artificial) medications require appropriate storage conditions. If not properly stored, these medicines may lose their ability to function correctly, completely, and with a lower efficacy. Medicines that treat minor ailments, e.g., minor aches and pains, may be used at a lower efficacy. However, medicines that treat serious medical conditions may always be used at their highest potency; otherwise, medicine may cause serious harmful effects for short-, long-term, or a combination of both. Although patients or care-takers are required to store the medicines under required environmental conditions, such conditions may not always be convenient and often not possible due to life-styles. As people leave their homes and travel either locally or long-distance, they pack the medicines in their luggage, backpacks, purses, briefcase, commuter bags, etc. The medicines may not be stored under required environmental conditions; hence, the medicines may become less potent and inefficient. Often the patients or the care-takers forget to carry the medicines, which adds to further health problems of missing dosages or medication not available when required. Epinephrine auto-injector is a life-saving medicine but may not be used daily; forgetting to carry the medication while traveling may be common. The forgetfulness to carry, and hence, administer the medicine may cause serious health problems and even death. To overcome these problems, the current application's inventors have created a safety device, systems, and methods to store and carry medicines under required temperature, pressure, humidity, light exposure, etc.

The list of medications that may be sensitive to environmental conditions is large and includes life-threatening and non-life-threatening. However, most common and well known environmental conditions sensitive medications may be injectable epinephrine, nitroglycerin, levothyroxine, insulin, epi-pen, asthma inhalers, opioid antagonists, insulin and glucagon., naloxone, rheumatoid arthritis injectables, maycer injectables, antibiotics, cardiac medications, nitroglycerin, anticoagulants, antiplatelet agents, ACE inhibitors, angiotensin II receptor blockers, angiotensin receptor-neprilysin inhibitors, beta blockers, calcium channel blockers, cholesterol lowering medications, digitalis preparations, diuretics, vasodilators, anticonvulsant/anti-seizure medications, testosterone, estradiol, progesterone, luteinising hormone, estrone, dehydroepiandrosterone, estriol, follicle-stimulating hormone, growth hormone, cortisol, testosterone undemayoate, androstenedione, human chorionic gonadotropin, norepinephrine, prostacyclin, NSAIDS, narcotics, opioids, arthritis medication, immunosuppressants, Analgesics, including opioids and non-opioids, Anesthetics, Antibacterial, including antibiotics, Anticonvulsants, Antidementia agents, Antidepressants, Antidotes and antitoxins, Antiemetics, Antifungals, Anti-inflammatory agents, including corticosteroids, Antimigraine agents, Antimyasthenic agents, Antimycobacterials, Antineoplastic, Antiparasitic, Antiparkinson agents, Antipsychotics, Antivirals, including HIV antiretrovirals and direct-acting hepatitis C drugs, Anxiolytic (anti-anxiety) agents, Bipolar agents, Blood glucose regulators, including insulin and other diabetes medications, blood products, including anticoagulants, cardiovascular agents, including beta-blockers and ACE inhibitors, central nervous system agents, including amphetamines, dental and oral agents, dermatological (skin) agents, enzyme replacement agent, gastrointestinal agents, including H₂ blockers and proton pump inhibitors, genitourinary (genital and urinary tract) agents, hormonal agents (adrenal), hormonal agents (pituitary), hormonal agents (prostaglandins), hormonal agents (sex hormones), including estrogen, testosterone, and anabolic steroids, hormonal agents (thyroid), hormone suppressant (adrenal), hormone suppressant (parathyroid), hormone suppressant (pituitary), hormone suppressant (sex hormones), hormone suppressant (thyroid) immunological agents, including vaccines and disease-modifying anti-rheumatic drugs (DMARDs), inflammatory bowel disease agents, metabolic bone disease agents, ophthalmic (eye) agents, otic (ear) agents, respiratory tract agents, including antihistamines and bronchodilators, sedatives and hypnotics, skeletal muscle relaxants, therapeutic nutrients, minerals, and electrolytes, etc., and any combination thereof.

In an embodiment of the invention, the device may be attached and/or detached to, but not limited to, the cellular phone, commuter case, backpack, belt, purse, luggage, briefcase, clothing, jewelry, or body parts such as wrists, arms, neck, waist, hospital equipment, ankles, legs, torso, specialized medical equipment, adaptive equipment, mobility adaptive equipment, wheelchair, crutches, prosthetic devices, orthotic devices, sensory adaptive equipment, assistive listening devices, augmentative and alternative communication devices, alerting devices, baby carrier, diaper bag, baby bag, duffle bag, gym bag, messenger bag, bike bag, trunk bag, saddle bag, fanny pack, hip pack, suspenders, gun sling, gun holster, gun cases, gun bags, tackle bags, tablet case, electronic case, instrument case, eyeglass case, glasses pouch/case, tool box, tool belt, tool bag, wallet, medical bag, first aid kit, pet bag, pet accessory bag, travel pet carrier, trunk, satchel, haversack, suitcase, randoseru, hobo bag, minaudiere, tote bag, tote bag, sling, clutch bag, baguette, laptop bag, security pouch, wash bag, dopp kit, weekender, barrel bag, bowling bag, safari bag, field bag, battle bag, pannier bag, camera bag, packing cubes, tennis bag, hiking backpack, golf bag, guitar bag, portfolio bag, garment bag, army duffle bag, coin purse, clasp purse, long wallet, zip around wallet, credit card wallet, bi-fold wallet, tri-fold wallet, wallet with chain, wallet with strap, passport holder, phone case wallet, neck wallet, taxi wallet, shoe wallet, leg wallet, wrist wallet, RFID (radio frequency identification devices), wallet hardside luggage, softside luggage, rigid case, cosmetic case, pilot case, camera hard case, daypack, belt bag, bum bag, waist bag, drawstring bag, rucksack, satchel backpack, knapsack, straw bag, muff bag, woven tote, fringe bag, bucket bag, accordion handbag, shopper bag, mayteen bag, quilted sling, lady camera bag, sling bag, key case, watch strap, card holder, writing case, checkbook cover, luggage tag, manicure set case, pencil case, stroller, adult stroller, walker, pushchairs, home care hospital beds, adjustable beds, patient lifts, patient slings, protective helmets, lift chair, scooter, bariatric and heavy duty medical equipment, pediatric bed, pediatric mattress, pediatric car seats, special needs car seats, bathroom aids, bath chairs, shower chair, commode chair, transfer benches, shower trolley, free standing commode, harness, net swing, therapy bench, floorsitter, classroom chair and classroom desk, recliner, rocking chair, activity seating, speech generating device, walking aids, gait trainer, rollator, adaptive brace, adaptive support, safety sleeper, beach wheelchair, dental equipment, adaptive driving equipment, safe, lock box, headband, key chain, bike, golf cart, a TV, motor vehicle, boat, airplane, jet, flashlight, television, dvd player, game consoles, fan, desktop computer, printer, radio, appliances, refrigerator, stove, washer, dryer, cabinets, wall-mounts, or/and any combination thereof.

In an embodiment of the invention, the medication may be used by babies, toddlers, children, teenagers, adults, seniors, pets, etc., and any combination thereof.

In an embodiment of the invention, the attachment may be temporary, semi-permanent, permanent, and/or any combination thereof.

In an embodiment of the invention, the attachment may be via, but not limited to, glue (permanent, semi-permanent, or temporary), magnets, the zipper, suction-cups, velcro, clips, micro-clips, foldable, quick side release buckles, sealer, clip clasp, spring hooks, snap, trigger snap clips, snap pens, button, buttons, toggles, studs, snap fasteners, poppers, eyelets, buckles, hook and loops, frogging, hooks and eyes, grommets, brooches, safety pens, fabric ties and laces, tape, invisible tape, stabilizing tape, an interlocking mechanism, slide lock closure, single zipper closure, double zipper closure, zipper closure, attaching mechanism suitable for application, detaching mechanism suitable for the application, lock, track system, sliding track mechanism, connecting mechanism, vacuum seal, air tight seal, zip tie, twist tie, press to open, press to close, open lid, close lid, twist on, twist off, lock and unlock mechanism, controlled lock, combination lock, key lock, etc., and/or any combination thereof.

In an embodiment of the invention, the medicine may be in one or more forms such as, but not limited to, solid, powder, liquid, semi-liquid, gel, aerosol, gaseous, and/or any combination thereof.

In an embodiment of the invention, the medicine may be delivered via one or more routes such as, but not limited to, oral, sublingual, intravenous, intramuscular, transdermal, anal, etc., and/or any combination thereof.

In an embodiment of the invention, the medicine may be one or more, but not limited to, chemicals, compounds, compositions, biologics, biosimilar, peptides, etc., and/or any combination thereof.

The device may be a receptacle configured to store one or more medicines safely, the receptacle comprising an outer jacket, one or more inner compartments and/or sub-compartments.

In an embodiment of the invention, the outer jacket may be made of one or more of synthetic and/or non-synthetic materials such as a number of non-synthetic or synthetic materials e.g., plastics, a combination polyvinyl chloride (PVC), moldable polymers, flexible polymers and plastics, acrylics, polyesters, silicones, polyurethanes, and halogenated plastics., hermoplastics and thermosets, conductive polymers, biodegradable plastics and engineering plastics, polyethylene (PE), polypropylene (PP), polystyrene (PS) and polyvinyl chloride (PVC), Polycarbonate (PC), High-density polyethylene (HDPE), Polypropylene (PP), Acrylonitrile butadiene styrene (ABS), Polycarbonate plus Acrylonitrile Butadiene Styrene (PC+A), Phenolics or phenol formaldehyde (PF), Polyetheretherketone (PEEK), Polysulfone, Polydiketoenamine (PDK), velvet, denim, polar fleece, chiffon, felt, corduroy, flannel, brocade, crepe, organza, terrycloth, taffeta, damask, muslin, tweed, chenille fabric, gauze, lawn cloth, plush, voile, charmeuse, poplin, gabardine, gingham, hessian fabric, batiste, chintz, bamboo textile, broadcloth, lame, georgette, challis, sateen, velour, madras, mohair, tartan, bengaline, organdy, fustian, dimity, habutai, percale, jeans, chino cloth, tickling, whipcord, alpaca fiber, bedford cord, baize, moleskin, cotton, cellulosic fiber/viscose, wool, silk, leather, bast fibers, experimental fabrics, wood, notions and hardware, nylon, polyester, thermoplastic polyurethane (TPU), rubber, foam neoprene, crushed neoprene, vulmayized rubber, elastic, etc., and/or any combination thereof.

In an embodiment of the invention, the first inner compartment may be preventing heat and thermal energy exchange between the medicine-containing compartment and the surrounding environment. This compartment may contain a vacuum and may be airtight. This compartment may be lined on inner, outer, and/or both by reflective materials such as silver, stainless steel, etc.

In an embodiment of the invention, the second compartment may provide heating or cooling to the device. This compartment may be filled with liquid nitrogen, a combination of dry-ice and alcohol such as but not limited to methanol, ethanol, propanol, etc., any refrigerants and freons, such as but not limited to chlorofluorocarbons (CFCs), Hydrochlorofluorocarbons (HCFCs), Hydrofluorocarbons (HFCs), and Natural Refrigerants, Chlorofluorocarbons (CFCs)—R11, R12, R113, R114, R115, Hydrochlorofluorocarbons (HCFCs)—R22, R123, Hydrofluorocarbons (HFCs)—R134a, R404a, R407C, R410a.

In an embodiment of the invention, the second compartment may be further divided into another sub-compartment for providing insertable heat or cooling and may be fully and/or partially hollow space for replaceable inserts. The inserts may be heating and/or cooling materials such as, but not limited to, exothermic-, endothermic-materials/reactants, heated or cooled gels, rice, plastic, polyethylene terephthalate (PET or PETE or polyester), high density polyethylene (HDPE), polyethylene terephthalate, thermoformed plastic, polyvinyl chloride, low density polyethylene (LDPE), polypropylene (PP), polystyrene (PS), polycarbonate (PC), Lexan, Makrolon, Makroclear, polyethylene, steel, carbon steel, alloy steel, stainless steel, iron, wrought or cast iron, aluminum, magnesium, copper, brass, bronze, zinc, titanium, tungsten, adamantium, nickel, cobalt, tin, lead, silicon, erbium, gold, hafnium, lead, americium, barium, berkelium, beryllium, bismuth, bohrium, cadmium, calcium, californium, cerium, chromium, curium, darmstadtium, dubnium, dysprosium, einsteinium, erbium, europium, fermium, francium, gadolinium, gallium, gold, hassium, holmium, indium, iridium, iron, lanthanum, lawrencium, lithium, lutetium, lutetium, magnesium, manganese, meitnerium, mendelevium, molybdenum, mercury, neptunium, niobium, nobelium, osmium, palladium, platinum, plutonium, polonium, potassium, praseodymium, promethium, radium, rhenium, rhodium, roentgenium, rubidium, ruthenium, rutherfordium, samarium, smaydium, seaborgium, silver, sodium, strontium, tantalum, technetium, terbium, tallium, thorium, thulium, ununbium, vanadium, ytterbium, yttrium, zinc, zirconium, ununhexium, ununpentium, ununquadium, ununtrium, metalloids, alkali metals, alkaline earth metals, transition metals, ceramics, spiders' silk, silicon carbide, nanospheres/nano kevlar, wurtzite boron nitride, lonsdaleite, dyneema, metallic glass, buckypaper, graphene, diamond, polyurethane foam, phenolic foam, carbon, copper, plastic film, transparent plastic embedded with glass spheres, glass powder, polymethylpentene, silver, “passive cooling” technology, silicon dioxide, hafnium dioxide, transparent plastic, radiative cooling technology, nanofluids, water, ethylene glycol, oil, cooling fluids, heat sinks, etc., or any combination thereof.

In an embodiment of the invention, the second compartment may be refillable, replaceable, insertable, sealed, unsealed, partially sealed, accessible, and/or any combination thereof.

In an embodiment of the invention, the third inner compartment may be reducing exchange energy via the use of an insulating material such as but not limited to fiberglass, R-3.1, mineral wool R-3.1, cellulose R-3.7, polystyrene extruded (XEPS), polyurethane, polyurethane foam, R-6.3 polystyrene (EPS), R4 aerogel, pyrogel, pyrogel XT, hemp, sheep's wool, cotton, straw, polyisocyanurate, Hexabromocyclododemaye (HBCD), fireproof fiberglass, polyurethane foam, phenolic foam, carbon, copper, plastic film, transparent plastic embedded with glass spheres, glass powder, polymethylpentene, silver, “passive cooling” technology, silicon dioxide, hafnium dioxide, transparent plastic, radiative cooling technology, nanofluids, water, ethylene glycol, oil, cooling fluids, heat sinks, air, thermal insulating barrier, gel ice pack, cool cell container, cryogenic technology, hydroxyethyl cellulose, sodium polyacrylate, vinyl coated silica gel, ammonium nitrate, calcium ammonium nitrate, urea, hot packs, cold packs, silica gel, propylene glycol, thickener, coloring, silicone-coated fiberglass, flame retardant material, air, etc., or any combination thereof.

In an embodiment of the invention, the device may use active cooling solutions, such as but not limited to, forced air through a fan or blower, forced liquid, and thermoelectric coolers (TECs), which may be used to optimize thermal management on all levels and passive cooling, solutions that such as heat sinks, heat spreaders, heat pipes or thermal interface materials (TIM) to maintain optimal temperatures.

The third compartment may be permanently sealed, temporarily sealed, unsealed, and/or any combination thereof.

In an embodiment of the invention, the fourth compartment may be humidifying or dehumidifying substances and/or any combination thereof. The fourth compartment may be sealed or not sealed.

In an embodiment of the invention, desiccants may be used to lower humidity. For example, but not limited to activated alumina, Aerogel, Benzophenone, Bentonite clay, Calcium chloride, Calcium oxide, Calcium sulfate (Drierite), Cobalt(II) chloride, Copper(II) sulfate, Lithium chloride, Lithium bromide, Magnesium chloride hexahydrate, Magnesium sulfate, Magnesium perchlorate, Molecular sieve, Phosphorus pentoxide, Potassium carbonate, Potassium hydroxide, Rice, Silica gel, Sodium, Sodium chlorate, Sodium chloride, Sodium hydroxide, Sodium sulfate, Sucrose, Sulfuric acid, etc., and/or any combination thereof.

In an embodiment of the invention, moisture may be used to increase humidity. The humidity may be entrapped in natural and/or synthetic chemicals. The humidity may be released slowly, quickly, customized released, and/or any combination thereof.

In an embodiment of the invention, the fifth compartment may be temperature, pressure, and/or humidity sensing via color-changing materials, the liquid crystal thermometer, and/or temperature-sensors, humidity-sensors. This compartment may be partitioned into two or more subcompartments such that one or more temperature, light, pressure, and humidity may be monitored.

In an embodiment of the invention, the fifth compartment may contain two or more sub-compartments to detect or sense temperature-, humidity-, pressure-, light-indicators may be color-changing humidity detecting chemicals, color-changing temperature detecting chemicals, sensors, micro-sensors, nano-sensors, etc., and a combination thereof.

In an embodiment of the invention, there may be several different thermochromic responses depending on the configuration. For example, the green color may represent a safe temperature range, the yellow color may represent a cautious temperature range, and the red color may be a mean warning out of range temperature(s). Similarly, another color scheme may be where color blue, purple, brown, dark blue, red, etc., may represent safe temperature(s) and colors white, red, blue-pink, pink, light blue, etc., means unsafe temperatures(s), and/or any combination thereof. These colors may be replaced by other visual, audio, tactile, etc., signals and/or any combination thereof.

In an embodiment of the invention, the temperature of the compartments and/or subcompartments may be monitored by using color-changing filament, 3D printer filament, thermochromic pigment, thermochromic paint, thermochromic plastics, thermochromic nail polish, thermochromic ink, thermochromic glitter, thermochromic liquids, thermochromic crystals, thermochromic dyes, thermochromic powders, leuco dyes, reversible thermochromic pigment, irreversible thermochromic pigment, color to colorless thermochromic pigment, color to colorless thermochromic pigment, digital, etc. The outer jacket may have a digital or non-digital display panel of the internal of the medicine/medicine storage compartment temperature, etc., and/or any combination thereof.

In an embodiment of the invention, the fifth compartment may be transparent, semi-transparent, translucent, opaque, and/or any combination thereof.

In an embodiment of the invention, the sixth compartment may be a storage unit for at least one medicine. The sixth compartment may be sealable, unsealable, partially sealable, closable, accessible mechanisms, and/or any combination thereof.

In an embodiment of the invention, the sixth compartments may further comprise one or more sub-compartment for two or more medicines.

In an embodiment of the invention, the sixth subcompartments may further comprise first, second, third, fourth, fifth compartments. These are referred to herein as subcompartments. These subcompartments may be permanently or temporarily sealed or not sealed.

In an embodiment of the invention, the sixth compartment or subcompartment may have compartments or subcompartments one through five adjust to and in-contact with top, bottom, front, back, right-, left-sides, or any combination thereof.

In an embodiment of the invention, the compartments and sub-compartments may be of equal size and/or unequal size, maybe in any combination of order, and may contain one or more compartments and sub-compartment. For example, in an embodiment, the device is in the order of innermost to outermost compartments as six, second, fourth, one, and third, and be missing compartment five.

In an embodiment of the invention, the temperature may be from minus 180° C. to minus 10° C. to 0° C. to plus 69° C. For example, −180, −170, −160, −150, −140, −130, −120, −110, −105, −104, −103, −102, −101, 0, +1, +2, +3, +4, +5, +6, +7, +8, +9, +10, +11, +12, +13, +14, +15, +16, +17, +18, +19, +20, +21, +22, +23, +24, +25, +26, +27, +28, +29, +30, +31, +32, +34, +35, +36, +37, +38, +39, +40, +45, +50, +55, +60, +61, +62, +63, +64, +65, +66, +67, +68, +69, +70, +71, +72, +73, +74, +75, degrees celcius etc., and/or combination thereof.

In an embodiment of the invention, the humidity may be from 20-60%. For example, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 percent and/or any combination thereof.

In an embodiment of the invention, the partition between the compartments may be solid, perforated, permeable, impermeable, membranes, and/or any combination thereof.

In an embodiment of the invention, the partition between the compartments may be light-reflective, energy absorptive, light non-reflective, energy non-absorptive membranes, and/or any combination thereof.

The device and system further may contain materials, coating, and methods for reflecting, not absorbing for all lights wavelengths such as, but not limited to, sunlight, artificial light, all UV lights, or a combination thereof.

The device and system may contain materials and methods for reflecting, not absorbing, all energy wavelengths.

The device and system may contain materials and methods for not reflecting, absorbing all energy wavelengths.

In an embodiment of the invention, the receptacle may be further configured to comprise sensors such as, but not limited to, detecting temperature, humidity, pressure, weight, mass, touch-sensitive sensors, etc., and/or any combination thereof.

In an embodiment of the invention, the device and system may be configured with communication platforms and may have GPS, micro-GPs, Bluetooth, RFID, WiFi, internet of things, etc., and/or any combination thereof.

In an embodiment of the invention, the device and system may be configured to comprise micro/nano-cooling, heating systems, and/or both.

In an embodiment of the invention, the device and system may be configured to comprise power sources such as batteries, renewable batteries, rechargeable-batteries, replace batteries, micro-batteries, etc.

In an embodiment of the invention, the device and systems may be configured to alert the user when the medication may be exposed to undesired conditions such as but not limited to temperature, humidity, pressure, light, etc., or any combination thereof.

In an embodiment of the invention, the device and systems may be configured to alert in real-time, near-time, customized, and/or any combination thereof.

In an embodiment of the invention, the device and systems may be configured to provide customized alerts, wherein the alerts may be set by the users and/or care-takers based on pre-set limits. For example, a user or take-care may set 0° C. as an alert to replace medicine, −4° C. may be sent as a cautionary alert to replace the cooling insert. The user will get a warning/alert as the storage medicine/storage compartment temperature changes. The alerts for all environmental conditions may be customizable by the user, care-taker, manufacturer, and any combination thereof. Alerts may be sent based upon pre-set and customized conditions for one or more environmental conditions such as temperature, pressure, light, humidity, etc., and/or any combination thereof.

In an embodiment of the invention, the device and system may be configured to alert the user via audio, visual, tactile, an APP, etc., and any combination thereof.

In an embodiment of the invention, a standard clamshell phone case may use a mechanical or magnetic attaching mechanism to hold the medication compartment. Wherein the mechanisms may be magnets, zippers, fasteners, latches, bolt, batten, screw, fastener, buckle, button, tie, chain, clamp cramp, clasps, lobster clasp, clips, clutch, pin, hook-and-eye closure, hook and loop fastener, Velcro, latch, nail, pegs, clothespin, cotter, dowel, linchpin, R-clip, split pin, spring pin, tapered pin, retaining rings, circlip, e-ring, rivet, rubber band, ribbons, snaps, staple, stitches, strap, etc., and any combination thereof.

In an embodiment of the invention, the medication compartment outer layers will be both soft and hard plastic and may have physical temperature indicators.

In an embodiment of the invention, a medication compartment will open and seal airtight to hold the medication, such as the Auvi-Q and/or any other medicines with similar size and dimensions.

In an embodiment of the invention, the inside of the medication compartment will likely need to be made of stainless steel in order to add vacuum insulation (an outer wall, insulation, then inner wall).

In an embodiment of the invention, the typical dimensions maybe about 6.9×3.7×1.8 inches, 2.5×1.5×0.375 inches, 8.25×5.25×1.4375 inches, 14×9×2.5 inches, 4″×2″×0.25 inches, about 10″×10″×0.25 inches, 17″ wide×8″ deep×15″ tall, and combination thereof.

In an embodiment of the invention, the device may weigh maybe 2 ounces to 9 ounces to 6 ounces to 1 lb.

In an embodiment of the invention, the device may use machine learning and artificial intelligence to gather usage data and send customizable alerts. The collected data may be used along with the training data to achieve customizable alerts.

### EXAMPLE 1

Dara Miles is the primary caregiver as a parent to her seven-year-old son, Miles Jr. Miles Jr. is allergic to peanuts, so his mother is responsible for carrying an Epinephrine auto-injector (EAI) in her purse. Because the mother is careful with Mile Jr's food, she rarely needs to inject the epinephrine into her son. Therefore, she often forgets to carry the EAI. Since the epinephrine is very temperature sensitive, she must routinely replace the EAI in her purse with a properly functioning EAI. Hence, even when she has the AEI with her, it may not be effectively functioning due to exposure to high temperatures in the car on a typical day. The best storage for epinephrine is 20-25° C. (68-77° F.); however, temperature between 15-30° C. (59-86° F.) may be in the cautionary range. The invention detail herein will assist her in carrying properly stored medication at the required temperature. Since she always takes her cellular phone with her, she is unlikely to forget the EAI with it.

In an embodiment of the invention, insulin may be the stored medicine and replace in the above example. Insulin may be maintained at a temperature of 68-77° F., with brief excursions permitted from 59-86° F.

In an embodiment of the invention, she can customize an alert system. When the temperature increases above the required temperature range, such as above 25° C., she may be notified by email, text message, a message in the APP, visual, audio, tactile, etc. signal. In an emergency anaphylactic event, she will be best prepared to save her son's life by the use of the device and systems of this invention.

### Detailed Description of Figures

Below is a detailed description of the figures. In an embodiment of the invention depicted in the figure, the example herein uses the medicine Auvi-Q and cellular phone the attached device. However, it is to be understood that the above description is intended for illustrative purposes and is not limited and restrictive to this example. Other embodiments of the invention may be possible with other medicines and attached to other daily-use devices.

FIG. 1: One View of the Storage Device: The storage device is attached or integrated into a foldable style 101. The phone-case may have a secure zip feature where you may unzip and zip to access and secure the contents inside the storage component 102. Flap closure-magnet, Velcro, snap, or similar may be added to secure flap closure to the overall phone case 103. The storage compartment may store medicine(s) such as Auvi-Q herein 104. Shown here are cell phone (not included) 105, cell phone holder 106, closure/sealing agent/lid/member 109, foldable phone case107, the dimension of 107 are dependable on accommodating the thickness of the storage compartment and phone. The flap closure meets here to secure phone case 108. The temperature-sensitive compartment with thermochromic liquid crystal is located at 110.

FIG. 2: Side View of Stage Device with Foldable Style. The storage device is attached or integrated into a foldable style 201. The phone case may have a secure zip feature where you may unzip and zip to access and secure the contents inside the storage component 202. Flap closure-magnet or Velcro may be added to secure flap closure to the overall phone case 203. Medicine may be stored, such as Epinephrine auto-injectors such as Auvi-Q and epi-pen, herein storage compartment 204. Shown herein is a cell phone holder 205, closure/sealing agent/lid/member 209, thermochromic liquid crystals thermometer 210, foldable area of the phone case 206, which helps accommodate the thickness of the storage compartment and phone 207. The flap closure (#3) meets here to secure phone case 208.

FIG. 3: Storage Device Attachment Option with Side View. Contents example, Auvi-Q, for visual purposes on (not included). Medicine is stored, such as Auvi-Q, herein storage compartment 304. Show herein is a cell phone (not included) 305, cell phone holder 306, thermochromic liquid crystals thermometer 310.

FIG. 4: A prototype of the device and system. Shown herein is an outermost surface of one embodiment of a prototype. The medicine storage device may have a temperature indicator for the medication or the medication containing compartment 401. The storage compartment must be adequately enclosed 403 within the compartment/subcomponent such that the medicine is minimally exposed to the environment and maybe appropriately latched 402.

FIG. 5: An assembly of compartment and sub-compartments. The compartments of inventions may be arranged in a variety of orders and show herein one possible manner/embodiment. The compartments and sub-compartments may be of exactly the same sizes and/or different sizes. The compartments and sub-compartments may be manufactured as one unit or as separate units or compartments and then connected. The connection may be by a variety of manners, such as but not limited to welding, glue, cement, adhesives, etc., and/or any combination thereof. In this embodiment of the invention, at least one medication is stored in compartment 507; this compartment and sub-compartment may be customized to fit at least one medication perfectly. The outermost jacket 501 may be placed in a cellular phone case's pocket; the outer jacket may be made of soft, hard, or both plastics. The outer jacket may be attached, detached, detachably attached, etc., to a smartphone case (not shown herein). If the medication requires extremely low temperatures, such as minus 120° C., compartment 504 may be filled with liquid nitrogen. All rules and regulations regarding liquid nitrogen safety will be followed. However, if the temperature required is not as extreme and is 20° C., this compartment 504 may contain a replaceable cooling insert. Compartment 605 may have insulation materials, 506 may contain desiccating materials to keep the device's overall humidity low. Compartment 502 may contain one or more sensors and/or sensing chemicals. This compartment may sense one or more physical conditions, such as temperature, humidity, pressure, light, etc., in the device and medicines. This compartment and/or its sub-compartment may contain any required electronics such as batteries, a small/micro-speaker for audible alerts, communication platforms, and smart platforms such as Bluetooth, WiFi, micro-WiFi, internet, etc. In an embodiment, the device may electronically connect/paired to the user's phone and may capture usage data for alerts such as but not limited to temperature alerts, no medicine in the storage device alerts, medicine forgot alert, the device is in an extreme environment alert, e.g., a hot car, rain, freezing temperature, etc. The electronic connection may be manually and/or wirelessly via Bluetooth, WiFi, micro-WiFi, internet, etc. The device may exchange, receive, send, share, etc., data with a smartphone while in the case or when far away. The data may be exchanged, send, receive, transmit, share, etc., manually and/or wirelessly through Bluetooth, WiFi, micro-WiFi, internet, etc. The data exchanged, received, send, transmitted, sharing may be uni- and/or bi-directional. The sensing may be displaced on the device or transmitted to an APP. The medication storage device may be detachable from the phone/iPhone/smartphone case. This may allow for an easier fit into personal belongings such as a pocket, purse, etc. The outer shell with which the medication compartment attaches maybe be detachably-attachable to any traditional smartphone case.

FIG. 6: Shown herein is Auvi q with possible dimensions, which may be reduced to 0.375″ thick×1.5″ wide and 2.5″ long.

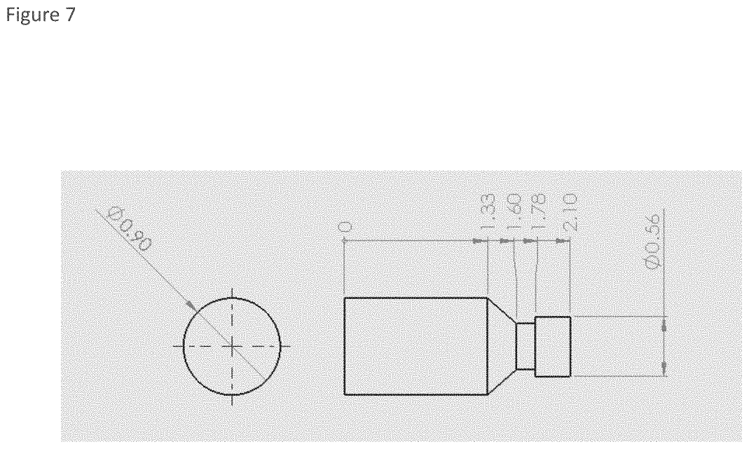

FIG. 7: Shown herein is a standard 10 mL medication vial.

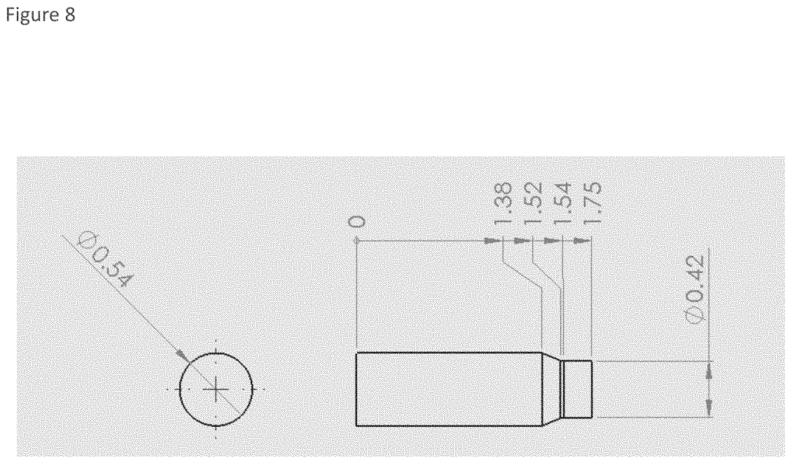

FIG. 8: Shown here is an aspect of one embodiment for a standard 3 mL medication vial for minimum size (attachment to an electronic device larger than 0.5″ in diameter and 1.5″ length).

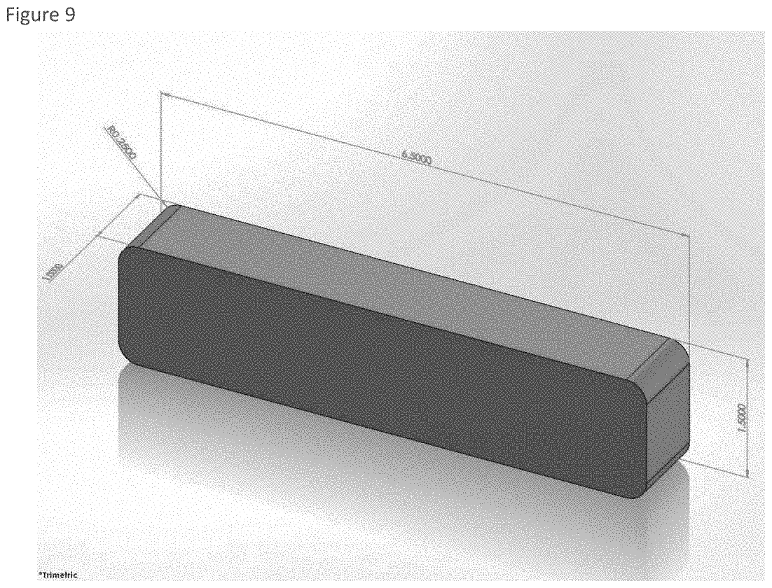

FIG. 9: Shown here is a case for an emergency glucose shot for diabetics 6.5″×1.5″×1″.

In an embodiment of the invention, the device and system may be manufactured in two or more parts or compartments and subcompartments such as a cellular phone holder, e.g., a thermoplastic polyurethane (TPU) case; stainless steel, a vacuum-insulated compartment along with a medication holding compartment., e.g., auto-injectors for epinephrine, insulin, glucagon, etc., such as epi-pen, Auvi-Q EAI, insulin pen, glucagon injection, etc. These may be auto-injector, prefilled syringe, injection vial, etc., but may be sensitive to environmental conditions, for emergency use, and needed to be carried for long or short travel, that is., may be required to be administered outside of the home.

In an embodiment of the invention, the two or more compartments and sub-compartments may be attached permanently or temporarily via glue/cement (permanent, semi-permanent, or temporary) such as a polycarbonate (PC) hard plastic component, magnets, the zipper, suction-cups, velcro, clips, micro-clips, foldable, quick side release buckles, sealer, clip clasp, spring hooks, snap, trigger snap clips, snap pens, button, buttons, toggles, studs, snap fasteners, poppers, eyelets, buckles, hook and loops, frogging, hooks and eyes, grommets, brooches, safety pens, fabric ties and laces, tape, invisible tape, stabilizing tape, an interlocking mechanism, slide lock closure, single zipper closure, double zipper closure, zipper closure, attaching mechanism suitable for application, detaching mechanism suitable for the application, lock, track system, sliding track mechanism, connecting mechanism, vacuum seal, air tight seal, zip tie, twist tie, press to open, press to close, open lid, close lid, twist on, twist off, lock and unlock mechanism, controlled lock, combination lock, key lock, etc., and/or any combination thereof.

There is a market for this invention due to the increased number of life-saving medications that may be sensitive to environmental conditions.

The market need for this invention is significant for the treatment of anaphylactic reactions due to allergies. Severe allergies and related growing health harms are growing. Allergic diseases are extremely common, affecting more than 20% of the population in developed countries. Food allergies are among the most common allergic diseases, affecting up to 550 million people worldwide and more than 26 million adults and 5 million children in the US. Among children with food allergies, more than 40% have gone to the emergency room at least once due to an allergic reaction. Globally, food allergies are on the rise, with a particularly troubling acceleration observed among older children and teens, who are at the highest risk of fatal anaphylaxis. Allergic reactions, which occur when the immune system responds inappropriately to a normally harmless substance, can range from mild to life-threatening and localized to systemic. The symptoms and severity of an allergic reaction can vary between people, and one's response to an allergen can change over time. The most serious form of allergic reaction is known as anaphylaxis. Anaphylaxis is unpredictable, costly, and life-threatening. Anaphylaxis is an acute, systemic, and potentially life-threatening hypersensitivity reaction. More than 48 million people in the US may be at risk of anaphylaxis. The symptoms of severe anaphylaxis include the rapid onset of the airway, breathing, circulatory, and/or gastrointestinal problems in many cases skin and mucosal symptoms such as hives, flushing, itching, and angioedema1. Because it can quickly progress to respiratory collapse, anaphylaxis can be fatal in fewer than 10 minutes without rapid treatment. There are many causes of anaphylaxis, but it most commonly results from IgE-dependent allergies, and the most common triggers include allergenic foods such as peanuts, tree nuts, fish, shellfish, milk, and eggs; medications; and insect stings. Though fatal anaphylaxis is rare (there were between 186 and 225 anaphylaxis-related deaths per year in the US from 1999-2009), its unpredictability requires vigilance by patients and caregivers to ensure that their EAIs are always within reach, so treatment can be administered within minutes of an anaphylactic episode. Anaphylaxis imposes a significant economic burden on patients and society, costing an estimated $1.2 billion each year in direct medical expenses in the US alone. Early administration of epinephrine is critical, which is facilitated by this invention.

It is to be understood that the above description is intended to be illustrative and not restrictive. For example, the above-described embodiments (and/or aspects thereof) may be used in combination with each other. In addition, many modifications may be made to adapt a particular situation, method, system device, or material to the teachings of the various embodiments of the invention without departing from their scope. While the particulars and details described herein are intended to define the parameters of the various embodiments of the invention, the embodiments are by no means limiting and are exemplary embodiments. Many other embodiments will be apparent to those of skill in the art upon reviewing the above description. The scope of the various embodiments of the invention should, therefore, be determined with reference to the appended claims, along with the full scope of equivalents to which such claims are entitled.

This written description uses examples to disclose the various embodiments of the invention, including the best mode, and also to enable any person skilled in the art to practice the various embodiments of the invention, including making and using any devices or systems and performing any incorporated methods. The patentable scope of the various embodiments of the invention is defined by the claims, and may include other examples that occur to those skilled in the art. Such other examples are intended to be within the scope of the claims if the examples have structural elements or steps that do not differ from the literal language of the claims, or if the examples include equivalent structural elements or steps with insubstantial differences from the literal language of the claims.

We claim:

1. A device configured to safely store at least one environment-sensitive medicine comprising a medicine-storage compartment configured to store at least one medicine, a vacuum compartment, an insulating compartment, at least one compartment with light-reflective materials, wherein the device is detachably attached to a smartphone case.

2. The device of claim 1, wherein the storage compartment is configured to stored medications selected from the group consisting of epinephrine, insulin, glucagon, and combination thereof.

3. The device of claim 1, wherein at least one compartment comprises exothermic materials

4. The device of claim 1, wherein at least one compartment comprises endothermic materials

5. The device of claim 1, wherein at least one compartment comprises humidity altering material.

6. The device of claim 1, wherein the device length is selected from a group consisting 2.5, 6.9, 8.25, 10, 14 4, 17 inches, the device width is selected from a group consisting of 1.5, 2, 3.7, 5.25, 9, 10, 15 inches, and the device depth is selected from a group consisting of 0.25, 0.375, 1.8, 1.4 5 2.5, 8 inches.

7. The device of claim 1, wherein at least one compartment is sealed and airtight.

8. The device of claim 1, wherein the partition between the compartments are selected from a group consisting of solid, perforated, membranes, permeable, impermeable, and any combination thereof.

9. The device of claim 1, wherein the environmental-sensitive conditions are selected from a group consisting of temperature, pressure, humidity, light, and any combination thereof.

10. The device of claim 1, wherein the temperature in the medicine storage compartment is maintained between minus 180° C. to minus 10° C. to 0° C. to plus 69° C.

11. The device of claim 1, wherein the detachably attached to the smartphone is selected from a group consisting of magnets, zippers, fasteners, latches, bolt, batten, screw, fastener, buckle, button, tie, chain, clamp cramp, clasps, lobster clasp, clips, clutch, pin, hook-and-eye closure, hook and loop fastener, Velcro, latch, nail, pegs, clothespin, cotter, dowel, linchpin, R-clip, split pin, spring pin, tapered pin, retaining rings, circlip, e-ring, rivet, rubber band, ribbons, snaps, staple, stitches, strap, and any combination thereof.

12. The device of claim 1, wherein the device outer jacket comprises soft and hard plastics.

13. The device of claim 12, wherein the out jacket has a displayed panel for the medicine storage compartment temperature.

14. The device of claim 1, wherein at least one compartment comprises stainless steel.

15. The device of claim 1, wherein the device comprises at least one insulation material.

16. The device of claim 1, wherein the device can wireless connection and communicate with a smartphone, wherein the device can wireless exchange data with the smartphone, wherein the data exchange is bi-directional.

17. The device of claim 1, wherein the device is configured to alert users, wherein the alerts are customizable with pre-set environmental conditions.

18. The device of claim 1, wherein the device is configured to alert the user when the medication storage compartment is exposed to unmet pre-set environmental conditions.

19. The device of claim 1, wherein the device can communicate an alert to the smartphone.

20. The device of claim 1, wherein the device is configured to alert the user selected from the group consisting of audio, tactile, an APP, and any combination thereof.
